(19) United States
(12) Patent Application Publication
(10) Pub. No.: US 2022/0323230 A1
(43) Pub. Date: Oct. 13, 2022

(54) SHOULDER PROSTHESIS WITH MODULAR EPIPHYSEAL PART

(71) Applicants: One Ortho, Saint.Genis.Laval (FR); Universite Savoie Mont Blanc, Chambery (FR); Franco-Swiss Orthoconsulting, Oberhausbergen (FR)

(72) Inventors: Pierre Vacher, Thorens Glieres (FR); Thibault Goyallon, Annecy (FR); Eric Vittecoq, Groisy (FR); Arnaud Dodelin, Bihorel (FR); Yvan Arlettaz, Monthey (FR); Benoit Schenck, Oberhausbergen (FR); Maxime Antoni, Strasbourg (FR); Christophe Alepee, Lyon (FR); Christophe Depres, Charvonnex (FR)

(21) Appl. No.: 17/616,068

(22) PCT Filed: Apr. 23, 2020
(86) PCT No.: PCT/FR2020/050688

§ 371 Date: Dec. 2, 2021

(30) Foreign Application Priority Data
Jun. 5, 2019 (FR) 1905951

(51) Int. Cl. A61F 2/40

(52) U.S. Cl. CPC A61F 2/4014; A61F 2002/3054

(57) ABSTRACT

The invention relates to a shoulder prosthesis comprising a diaphyseal part extending along a diaphyseal axis and on which an epiphyseal part is mounted, the epiphyseal part having an upper face which is inclined along a front plane with respect to the diaphyseal axis, and a lower central face bearing against a complementary upper face of the diaphyseal part. According to the invention, the epiphyseal part is reversible by rotation through 180° about an axis normal to the lower central face bearing against the epiphyseal part to modify the inclination of the upper face of the epiphyseal part.

### TECHNICAL FIELD

The present invention relates to the technical field of orthopaedic surgery, and more specifically, to the field of total shoulder prostheses.

The invention relates to a shoulder prosthesis with a modular epiphyseal part.

### PRIOR ART

A shoulder prosthesis with a modular epiphyseal part is completely known to a person skilled in the art.

Generally, and such as illustrated by document FR 2 876 899, this type of shoulder prosthesis comprises a diaphyseal part extending along a diaphyseal axis, surmounted by the epiphyseal part.

The epiphyseal part has an upper face inclined along a front plane with respect to the diaphyseal axis, and a lower central face bearing against a complementary upper face of the diaphyseal part.

In a known manner, the inclination of the upper face of the epiphyseal part varies according to the different types of implants. In most cases, this inclination has a smaller angle for an anatomical implant model, and a larger angle for an inverted implant.

As an example, for an anatomical shoulder prosthesis, the upper face of the epiphyseal part is inclined such that an axis normal to this face is itself inclined by an angle of 135° along the front plane and relative to the diaphyseal axis, while, for an inverted shoulder prosthesis, the axis normal to the upper face of the epiphyseal part is inclined by an angle of 145° along the front plane and with respect to the diaphyseal axis.

In other words, in an inverted shoulder prosthesis, the upper face of the epiphyseal part is closer to the horizontal than for an anatomical implant, which then makes it possible to improve the kinematics of the inverted shoulder prosthesis, while being more conservative of the bone stock of the patient since it is necessary to cut fewer bone parts internally and at the bottom.

This variability between the inclinations of the upper faces of the epiphyseal parts then multiplies the number of implants necessary to carry out the different surgical operations.

For example, with one-part shoulder prostheses, considering two possible angle values, namely for example 135° and 145°, for 4 epiphyseal sizes, and for 4 diaphyseal diameters, it is necessary to provide 32 humeral shoulder prostheses to respond to all possible configurations.

In order to overcome this disadvantage, and to decrease the number of implants required, it is well known to design shoulder prostheses with a modular epiphyseal part, as illustrated by patent FR 2 652 498.

According to this state of the art, the diaphyseal part then becomes common for the different types of prostheses, anatomical or inverted, which simplifies the manufacture of prostheses.

However, if the two angle values are always considered, namely for example 135° and 145°, 4 epiphyseal sizes and 4 diaphyseal diameters, it is necessary to provide 8 epiphyseal parts and 4 diaphyseal parts to meet all possible configurations, such that it is still necessary to design 12 parts.

Another solution exists to reduce this number of parts, which consists in particular of common diaphyseal parts between the anatomical and inverted prostheses and, in the case of the inverted prosthesis, to design wedge-shaped high-density polyethylene inserts which compensate for the difference in inclination between the different versions.

Thus, if the two possible angle values are considered, namely for example 135° and 145°, 4 epiphyseal sizes and 4 diaphyseal diameters, it is still necessary to provide 16 parts to respond to all possible configurations.

Moreover, the latter solution also has the disadvantage of requiring a greater bone section in the case of inverted prostheses, and a much greater stress on the insert at the bottom, also resulting in a greater stress on the fixing of the insert on its plate.

### DISCLOSURE OF THE INVENTION

One of the aims of the invention is thus to overcome the abovementioned disadvantages by proposing a humeral shoulder prosthesis with a modular epiphyseal part, making it possible to produce so-called anatomical prostheses, for example with an inclination of the upper face of the epiphyseal part of 135°, and a so-called inverted prosthesis, with an inclination for example of 145°, while reducing to the maximum the number of parts necessary to cover all the possible configurations, namely to provide, for example, different epiphyseal sizes and different epiphyseal diameters.

To this end, a shoulder prosthesis has been developed, and in a known manner, comprising a diaphyseal part extending along a diaphyseal axis, surmounted by an epiphyseal part. The epiphyseal part has an upper face inclined along a front plane with respect to the diaphyseal axis, and a lower central face bearing against a complementary upper face of the diaphyseal part.

According to the invention, the epiphyseal part is reversible by rotation through 180° about an axis normal to the lower central bearing face of the epiphyseal part, in order to modify the inclination of the upper face of the epiphyseal part, in particular between its two possible positions.

Thus, with the same epiphyseal part, it is possible to produce two shoulder prostheses, with different angles of inclination of the upper face of the epiphyseal part.

Thus, if two possible angle values are considered for the upper face of the epiphyseal part, 4 epiphyseal sizes and 4 diaphyseal diameters, it is therefore necessary to make available 4 parts for the epiphyseal parts and 4 parts for the diaphyseal parts, to meet all the possible configurations, i.e. a total of 8 parts.

The number of parts required is therefore reduced, which consequently reduces the manufacturing cost and the cost of the stock necessary for carrying out such an intervention.

According to one particular embodiment, the epiphyseal part is capable of adopting a first position in which an axis normal to its upper face is inclined by an angle of 135° along the front plane with respect to the diaphyseal axis, a second position, pivoted by 180° about the axis normal to its lower central bearing face, in which the axis normal to its upper face is inclined by an angle of 145° along the front plane with respect to the diaphyseal axis.

According to a particular embodiment, the lower central face of the epiphyseal part and the complementary upper face of the diaphyseal part are inclined by an angle a along the front plane with respect to the upper face, so as to make it possible to modify the inclination between the upper face of the epiphyseal part and the diaphyseal axis by an angle equal to +/−2α.

For example, if the angle a is equal to 5°, and the normal axis of the lower face of the epiphyseal part is inclined by an angle of 140° along the front plane and with respect to the diaphyseal axis, this allows the normal axis of the upper face of the epiphyseal part to be, in a first position, inclined by an angle of 135° and, in a second position, inclined by an angle of 145°.

### BRIEF DESCRIPTION OF THE DRAWINGS

Other characteristics and advantages of the invention will become apparent from the following description, which is given by way of non-limiting indication and made with reference to the appended figures, in which:

FIG. 1 is a schematic representation illustrating, at a front plane, a shoulder prosthesis according to the invention, the epiphyseal and diaphyseal parts being represented before assembly and to form an anatomical prosthesis;

FIG. 2 is a schematic representation similar to that of FIG. 1, illustrating the assembled epiphyseal and diaphyseal parts;

FIG. 3 is a schematic representation similar to that of FIG. 1, before assembly, to form an inverted shoulder prosthesis;

FIG. 4 is a schematic representation similar to that of FIG. 3, illustrating the assembled epiphyseal and diaphyseal parts.

### DETAILED DESCRIPTION OF THE INVENTION

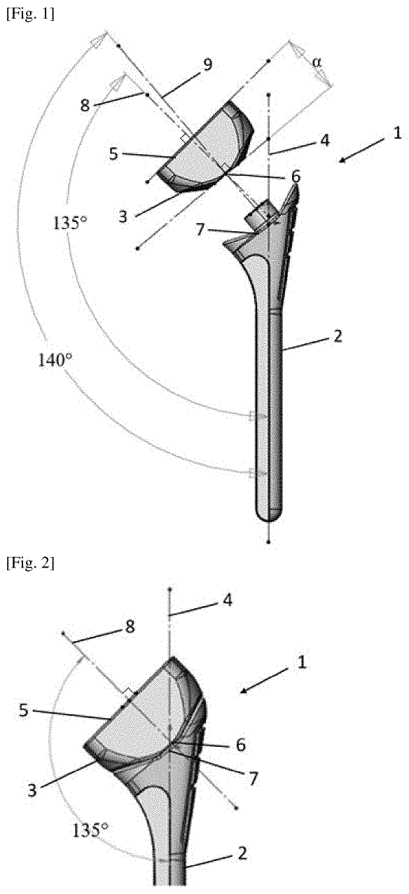

In reference to FIGS. 1 to 4, the invention relates to a modular shoulder prosthesis (1), i.e. that it comprises a diaphyseal part (2), and an epiphyseal part (3), which are distinct and can be interchanged.

In a known manner, the diaphyseal part (2) extends along a diaphyseal axis (4), and is surmounted by the epiphyseal part (3). The assembly between the diaphyseal and epiphyseal parts is well known to a person skilled in the art, such that it will not be described in detail.

Still in a known manner, the epiphyseal part (3) has an upper face (5) inclined along a front plane with respect to the diaphyseal axis (4), and a lower central face (6) bearing against a complementary upper face (7) of the diaphyseal part (2).

The inclination of the upper face (5) of the epiphyseal part (3) depends on the type of shoulder prosthesis.

For example, for a shoulder prosthesis of the anatomical type, the upper face (5) of the epiphyseal part (3) must be more inclined than for a prosthesis of the inverted type in which the upper face (5) of the epiphyseal part (3) is close to the horizontal.

In practice, the angle of inclination of the upper face (5) of the epiphyseal part (3) is defined by the angle formed by an axis (8) normal to said upper face (5) with respect to the diaphyseal axis (4).

The shoulder prosthesis (1) according to the invention makes it possible, with only one and same epiphyseal part (3), to design two shoulder prostheses (1), the upper faces (5) of the epiphyseal parts (3) of which have different angles of inclination.

To this end, and according to the invention, the epiphyseal part (3) is reversible by rotation through 180° about an axis (9) normal to the lower central bearing face (6) of the epiphyseal part (3) in order to modify the inclination of the upper face (5) of the epiphyseal part (3).

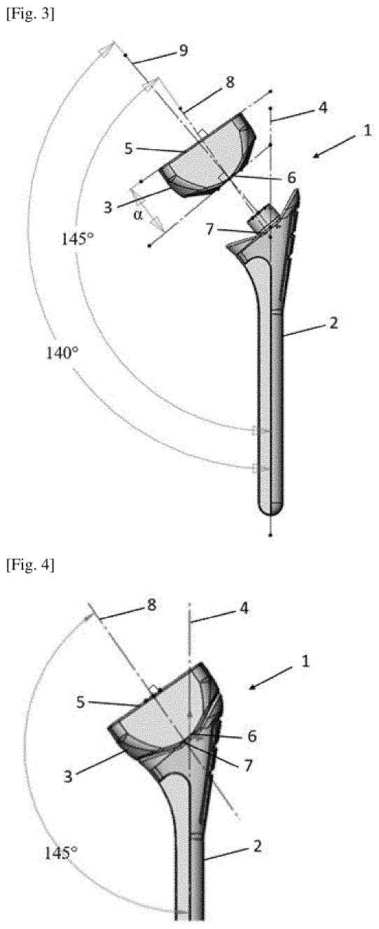

In other words, the epiphyseal part (3) is capable of adopting a first position (see FIGS. 1 and 2) in which the axis (8) normal to its upper face (5) is inclined by a first angle, for example 135°, along the front plane with respect to the diaphyseal axis (4), and a second position (see FIGS. 3 and 4) pivoted by 180° about the axis normal (9) to its lower central bearing face (6), in which the axis normal (8) to its upper face (5) is inclined by a second angle, for example 145°, along the front plane with respect to the diaphyseal axis (4).

In this way, by simple rotation of the epiphyseal part (3), it is possible to produce two different types of shoulder prostheses (1), for example a prosthesis (1) of the anatomical type, see FIGS. 1 and 2, and a prosthesis (1) of the inverted type, see FIGS. 3 and 4.

To do this, and according to a particular embodiment, the lower central face (6) of the epiphyseal part (3) and the complementary upper face (7) of the diaphyseal part (2) are inclined by an angle a along the front plane with respect to the upper face (5), so as to make it possible, during the pivoting of the epiphyseal part (3) by 180°, to vary the inclination between the upper face (5) of the epiphyseal part (3) and the diaphyseal axis (4) by an angle equal to +/−2α.

For example, in reference to the appended figures, the lower central face (6) of the epiphyseal part (3) and the complementary upper face (7) of the diaphyseal part (2) are inclined at an angle of 5° along the front plane and with respect to the upper face (5).

Thus, in the example of the figures, the axis (9) normal to the lower central face (6) of the epiphyseal part (3) is inclined along the front plane with respect to the diaphyseal axis (4) by an angle of 140°. In this way, the upper face (5) of the epiphyseal part (3) is capable of adopting a first position, see FIGS. 1 and 2, in which the axis (8) normal to said upper face (5) is inclined along the front plane and with respect to the diaphyseal axis (4) by an angle of 135° , and a second position, see FIGS. 3 and 4, in which the axis (8) normal to the upper face (5) of the epiphyseal part (3) is inclined along the front plane and with respect to the diaphyseal axis (4) by an angle of 145°.

From the above, if two possible angle values are considered, as mentioned above, namely 135° and 14°, 4 epiphyseal sizes and 4 diaphyseal diameters, it is then necessary to make available 4 parts for the epiphyseal parts (3) and 4 parts for the diaphyseal parts (2) to respond to all the possible implantation configurations, i.e. a total of 8 parts.

From the above, the present invention makes it possible to decrease the number of parts necessary for the design of different shoulder prostheses (1).

Of course, the values of the angles of inclination described above are not limiting. It can absolutely be considered to change the value of the angle a and/or the angle of inclination of the lower central face (6) of the epiphyseal part (3) and of the complementary upper face (7) of the diaphyseal part (2) to provide other types of prostheses (1) having other inclination, with, for example, pairs of angle values of 130°/140° or 140°/150°.

1. A shoulder prosthesis comprising a diaphyseal part extending along a diaphyseal axis, surmounted by an epiphyseal part , the epiphyseal part having an upper surface inclined along a front plane with respect to the diaphyseal axis , and a lower central face bearing against an upper complementary surface of the diaphyseal part, wherein the epiphyseal part is reversible by rotation through 180° about an axis normal to the lower central face of the epiphyseal part to modify the inclination of the upper surface of the epiphyseal part.

2. The shoulder prosthesis according to claim 1, wherein the epiphyseal part is adapted to adopt a first position in which an axis normal to its upper face is inclined by an angle of 135° along the front plane with respect to the diaphyseal axis, and a second position pivoted by 180° about the axis normal to its lower central face , in which the axis normal to its upper face is inclined by an angle of 145° along the front plane with respect to the diaphyseal axis.

3. The shoulder prosthesis according to claim 1, wherein the lower central face of the epiphyseal part and the complementary upper face of the diaphyseal part are inclined by an angle a along the front plane with respect to the upper face, so as to make it possible to modify the inclination between the upper face of the epiphyseal part and the diaphyseal axis by an angle equal to +/−2α.

4. The shoulder prosthesis according to claim 3, wherein the angle a is equal to 5°.

5. The shoulder prosthesis according to claim 2, wherein the lower central face of the epiphyseal part and the complementary upper face of the diaphyseal part are inclined by an angle a along the front plane with respect to the upper face, so as to make it possible to modify the inclination between the upper face of the epiphyseal part and the diaphyseal axis by an angle equal to +/−2α.

6. The shoulder prosthesis according to claim 5, wherein the angle a is equal to 5°.
